(19) United States
(12) Patent Application Publication
(10) Pub. No.: US 2025/0051623 A1
(43) Pub. Date: Feb. 13, 2025

(54) PROCESS FOR THE PREPARATION OF A BONDING RESIN

(71) Applicant: Stora Enso OYJ, Helsinki (FI)

(72) Inventors: Ashar Zafar, Älta (SE); David Almqvist, Tyresö (SE); Dimitri Areskogh, Bromma (SE); Huynh Tram Anh Pham, Älvsjö (SE)

(73) Assignee: Stora Enso OYJ, Helsinki (FI)

(21) Appl. No.: 18/717,644

(22) PCT Filed: Dec. 19, 2022
(86) PCT No.: PCT/IB2022/062447

§ 371 Date: Jun. 7, 2024

(30) Foreign Application Priority Data
Dec. 21, 2021 (SE) 2151566-3

(51) Int. Cl. C09J 197/00; B32B 5/02; B32B 21/02; C09J 5/00; C09J 11/04

(52) U.S. Cl. CPC C09J 197/005; C09J 5/00; C09J 11/04; B32B 5/02; B32B 21/02; B32B 2307/304

(57) ABSTRACT

The present invention relates to a bonding resin useful for example in the manufacture of laminates, mineral wool insulation and wood products such as plywood, oriented strandboard (OSB), laminated veneer lumber (LVL), medium density fiber-boards (MDF), high density fiberboards (HDF), parquet flooring, curved plywood, veneered particleboards, veneered MDF or particle boards. The bonding resin is also useful for example in composites, molding compounds and foundry applications. The invention also relates to a method for preparing the bonding resin. The bonding resin comprises an aqueous solution comprising lignin and/or tannin and ammonia and/or an organic base and hydroxymethylfurfural, furfural, furfuryl alcohol, acetoxy methyl furfural or an oligomer of hydroxy methylfurfural or a combination thereof and optionally one or more crosslinker and optionally one or more additives.

### FIELD OF THE INVENTION

The present invention relates to a bonding resin useful for example in the manufacture of laminates, mineral wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The bonding resin is also useful for example in composites, molding compounds and foundry applications. The invention also relates to a method for preparing the bonding resin.

### BACKGROUND

Lignin, an aromatic polymer is a major constituent in e.g. wood, being the most abundant carbon source on Earth second only to cellulose. In recent years, with development and commercialization of technologies to extract lignin in a highly purified, solid and particularized form from the pulp-making process, it has attracted significant attention as a possible renewable substitute to primarily aromatic chemical precursors currently sourced from the petrochemical industry.

Lignin, being a polyaromatic network has been extensively investigated as a suitable substitute for phenol during production of phenol-formaldehyde adhesives. These are used during manufacturing of laminate and structural wood products such as plywood, oriented strand board and fiberboard. During synthesis of such adhesives, phenol, which may be partially replaced by lignin, is reacted with formaldehyde in the presence of either basic or acidic catalyst to form a highly cross-linked aromatic resins termed novolacs (when utilizing acidic catalysts) or resoles (when utilizing basic catalysts). Currently, only limited amounts of the phenol can be replaced by lignin due to the lower reactivity of lignin.

One problem when preparing resins comprising lignin is the use of formaldehyde, when the lignin is used in formaldehyde-containing resins, such as lignin-phenol-formaldehyde resins. Formaldehyde based resins emit formaldehyde, which is a toxic volatile organic compound. The present and proposed legislation directed to the lowering or elimination of formaldehyde emissions have led to the development of formaldehyde free resin for wood adhesive applications.

Jingxian Li R. et al. (Green Chemistry, 2018, 20, 1459-1466) describes preparation of a resin comprising glycerol diglycidyl ether and lignin, wherein the lignin is provided in solid form. One problem with the technology described in the article is a long pressing time and high pressing temperature. The 3 plies plywood sample was pressed at 150° C. temperature for 15 minutes to fully cure the resins.

Engelmann G. and Ganster J. (Holzforschung, 2014, 68, 435-446) describes preparation of a biobased epoxy resin with low molecular weight kraft lignin and pyrogallol, wherein the lignin component consists of an acetone extraction from Kraft lignin.

### SUMMARY OF THE INVENTION

It has now surprisingly been found that it is possible to easily prepare a bonding resin in which the use of formaldehyde can be avoided. It has also been found that an improved bonding resin can be achieved by providing lignin and/or tannin in the form of an aqueous solution comprising ammonia and/or an organic base. By providing the lignin and/or tannin in the form of an aqueous solution comprising ammonia and/or an organic base, the step of milling of lignin and/or tannin particles can be avoided, avoiding lignin and/or tannin lump formation and the use of dispersing agent.

It has been found that when lignin and/or tannin is provided in the form of an aqueous solution comprising ammonia and/or organic base, the phenolic hydroxyl groups in the lignin and/or tannin structure are deprotonated and free to react with other components of a bonding resin. This improves the reactivity and performance of the binder. Therefore, providing the lignin and/or tannin in the form of a an aqueous solution comprising ammonia and/or an organic base speeds up the reaction significantly and hence reduces the pressing time and enables the use of a lower pressing temperature for curing the bonding resin, when manufacturing for example laminates, mineral wool insulation, glass wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The bonding resin is also useful for example in composites, molding compounds and foundry applications.

Furthermore, by providing lignin and/or tannin in the form an aqueous solution comprising lignin and/or tannin and ammonia and/or an organic base, the risk of degrading for example glass wool and mineral wool fibers is minimized.

Further, it has been found that by using hydroxymethylfurfural (HMF) or an oligomer thereof, furfural (FU), furfuryl alcohol (FA), acetoxymethyl furfural or an oligomer of HMF or a combination thereof, a bio-based thermoset binder can be achieved.

The present invention is thus directed to a method for preparing a bonding resin, wherein an aqueous solution comprising lignin and/or tannin and ammonia and/or an organic base is mixed with hydroxymethylfurfural (HMF), furfural (FU), furfuryl alcohol (FA), acetoxymethyl furfural or an oligomer of HMF or a combination thereof and optionally one or more crosslinker selected from glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound (such as tannin, cardanol, cardol, anacardic acid) or epoxidized plant-based oil (such as rapeseed oil, linseed oil, soy bean oil), tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, and/or diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate and optionally one or more additives.

The present invention is thus also directed to the bonding resin obtainable using the method described herein and to the use of the bonding resin in the manufacture of laminates, mineral wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The present invention is also directed to such laminates, mineral wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards manufactured using the bonding resin. The bonding resin according to the present invention may also be used in the manufacture of composites, molding compounds and foundry applications.

Thus, one aspect of the present invention is a bonding resin comprising an aqueous solution comprising lignin and/or tannin and ammonia or an organic base and hydroxymethylfurfural (HMF), furfural (FU), furfuryl alcohol (FA), acetoxymethyl furfural or an oligomer of HMF or a combination thereof and optionally one or more crosslinker selected from glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, and/or diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate; and optionally one or more additives.

### DETAILED DESCRIPTION

It is intended throughout the present description that the expression “lignin” embraces any kind of lignin, e.g. lignin originated from hardwood, softwood or annular plants. Preferably the lignin is an alkaline lignin generated in e.g. the Kraft process. Preferably, the lignin has been purified or isolated before being used in the process according to the present invention. The lignin may be isolated from black liquor and optionally be further purified before being used in the process according to the present invention. The purification is typically such that the purity of the lignin is at least 90%, preferably at least 95%. Thus, the lignin used according to the method of the present invention preferably contains less than 10%, preferably less than 5% impurities. The lignin may then be separated from the black liquor by using the process disclosed in WO2006031175. The lignin may then be separated from the black liquor by using the process referred to as the LignoBoost process. The lignin may be provided in the form of particles, such as particles having an average particle size of from 50 micrometers to 500 micrometers.

The tannin used in the present invention is a general term of complicated aromatic compounds having a large number of phenolic hydroxyl groups. Tannins are widely distributed in the plant kingdom, and to roughly divide, tannin is divided into two kinds of a hydrolyzed type and a condensed type. Both kinds are natural compounds, and have different structures. Either tannin may be used in the present invention. Polyhydric phenol compounds having a dye-fixing effect and a tanning effect of leather are called “synthetic tannin” and “cintan”, and among the synthetic tannins, the compounds which are effectively used can be used as well in the present invention.

The hydroxymethylfurfural (HMF), furfural (FU), furfuryl alcohol (FA), acetoxymethyl furfural or an oligomer of HMF or a combination thereof is preferably provided in liquid form, preferably as an aqueous solution. The weight ratio between lignin (dry weight) and/or tannin and the total amount of hydroxymethylfurfural (HMF), furfural (FU), furfuryl alcohol (FA), acetoxymethyl furfural or an oligomer of HMF, is preferably in the range of from 0.1:10 to 10:0.1, such as from 1:10 to 10:0.3, such as from 5:10 to 5:0.3, such as from 1:10 to 10:1. The amount of lignin and/or tannin in the bonding resin is preferably from 5 wt-% to 50 wt-%, calculated as the dry weight of lignin and/or tannin and the total weight of the bonding resin. In one embodiment, the aqueous solution comprising hydroxymethylfurfural (HMF), furfural (FU), furfuryl alcohol (FA), acetoxymethyl furfural or an oligomer of HMF also comprises base. Preferably, hydroxymethylfurfural is used according to the present invention. HMF oligomers are compounds having at least two linked HMF units/monomers. HMF oligomers preferably have a molar mass up to 3000 g/mol. HMF oligomers can be prepared according to methods known in the art, for example through a polycondensation.

The glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound (such as tannin, cardanol, cardol, anacardic acid) or epoxidized plant-based oil (such as rapeseed oil, linseed oil, soy bean oil), tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, and/or diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate that is optionally used according to the present invention acts as a cross-linker. Glycidyl ethers with more functional epoxide groups can be used such as glycerol diglycidyl ether, glycerol triglycidyl ether and sorbitol polyglycidyl ether. Other glycidyl ethers having two to nine alkylene glycol groups (such as 2-4 alkylene glycol groups or 2-6 alkylene glycol groups) can be used, such as diethylene glycol diglycidyl ether, triethylene glycol diglycidyl ether, dipropylene glycol diglycidyl ether and tripropylene diglycidyl ether. Other suitable crosslinkers include crosslinkers having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate and polyglycidyl methacrylate. Typically, the bonding resin according to the present invention is and applied to the surfaces of for example veneers, such as in the manufacture of plywood. When the veneers are pressed together under heating, the cross-linking in the bonding resin takes place, resulting in an adhesive. The hydroxymethylfurfural (HMF), furfural (FU), furfuryl alcohol (FA), acetoxymethyl furfural or an oligomer of HMF or a combination thereof provides a cross-linking effect.

In one embodiment of the present invention, the epoxy-based cross-linker, if used, has an epoxy index above 4 eq/kg. The epoxy index can be determined according to ISO 3001. Preferably, the cross-linker has an epoxy index above 5 eq/kg.

In one embodiment of the present invention, epoxy-based cross-linker is not used in the bonding resin.

An aqueous solution comprising lignin and/or tannin, further comprising ammonia and/or an organic base can be prepared by methods known in the art, such as by mixing lignin and ammonia and/or organic base with water. The pH of the aqueous solution comprising lignin and/or tannin comprising ammonia and/or an organic base is preferably in the range of from 8 to 14, more preferably in the range of from 10 to 14. Examples of organic bases include amines, such as primary, secondary and tertiary amines and mixtures thereof. Preferably, the organic base is selected from the group consisting of methylamine, ethylamine, propylamine, butylamine, ethylenediamine, methanolamine, ethanolamine, aniline, cyclohexylamine, benzylamine, dimethylamine, diethylamine, dipropylamine, dibutylamine, dimethanolamine, diethanolamine, diphenylamine, phenylmethylamine, phenylethylamine, dicyclohexylamine, piperazine, imidazole, 2-methylimidazole, 2-ethylimidazole, 2-ethyl-4-methylimidazole, 2-isopropylimidazole, 2-phenylimidazole, 2-methylimidazoline, 2-phenylimidazoline, trimethylamine, triethylamine, dimethylhexylamine, N-methylpiperazine, dimethylbenzylamine, aminomethyl propanol, tris(dimethylaminomethyl) phenol and dimethylaniline or mixtures thereof. The total amount of ammonia and/or organic base in the aqueous solution is preferably in the range of from 0.1 wt-% to 20 wt-%, preferably 0.1 wt-% to 10 wt-%, of the total weight of the aqueous solution comprising water, lignin and ammonia and/or an organic base. The amount of lignin and/or tannin in the aqueous solution of lignin and/or tannin, further comprising ammonia and/or an organic base is preferably from 1 wt-% to 60 wt-% of the solution, such as from 10 wt-% to 30 wt-% of the solution. The aqueous solution of lignin and/or tannin comprising ammonia and/or an organic base comprises less than 1 wt-% alkali and less than 1 wt-% inorganic base. More preferably, the aqueous solution of lignin comprising ammonia and/or an organic base does not comprise alkali and does not comprise inorganic base.

Thus, in a bonding resin according to the present invention the lignin and/or tannin is dissolved.

In one embodiment, the aqueous solution of lignin and/or tannin is a lignin solution, i.e. it does not comprise tannin. In one embodiment, the aqueous solution of lignin and/or tannin is a tannin solution, i.e. it does not comprise lignin. In one embodiment, the aqueous solution of lignin and/or tannin comprises from 5-95 wt % lignin and from 5-95 wt % tannin.

The weight ratio between lignin and/or tannin (dry weight) and the total amount of crosslinker, if used, is preferably in the range of from 0.1:10 to 10:0.1, such as from 1:10 to 10:0.3, such as from 5:10 to 5:0.3, such as from 1:10 to 10:1. The amount of lignin and/or tannin in the bonding resin is preferably from 5 wt-% to 50 wt-%, calculated as the dry weight of lignin and/or tannin and the total weight of the bonding resin.

The solid content of the bonding resin is preferably in the range of from 10 to 70%, such as in the range of from 15 to 50%.

The bonding resin may also comprise additives, such as urea, tannin, surfactants, dispersing agents and fillers. The bonding resin may also comprise plasticizer. As used herein, the term “plasticizer” refers to an agent that, when added to lignin, makes the lignin softer and more flexible, to increase its plasticity by lowering the glass transition temperature (Tg) and improve its flow behavior. Examples of plasticizers include polyols, alkyl citrates, organic carbonates, phthalates, adipates, sebacates, maleates, benzoates, trimellitates and organophosphates. Polyols include for example polyethylene glycols, polypropylene glycols, glycerol, diglycerol, polyglycerol, butanediol, sorbitol and polyvinyl alcohol. Alkyl citrates include for example triethyl citrate, tributyl citrate, acetyl triethyl citrate and trimethyl citrate. Organic carbonates include for example ethylene carbonate, propylene carbonate, glycerol carbonate and vinyl carbonate. Further examples of plasticizers include polyethylene glycol ethers, polyethers, hydrogenated sugars, triacetin and solvents used as coalescing agents like alcohol ethers. In one embodiment of the present invention, the plasticizer is a polyol, such as a polyol selected from the group consisting of polyethylene glycols and polypropylene glycols. The weight ratio between plasticizer and lignin and/or tannin, calculated on the basis of dry weight of each component, is from 0.1:10 to 10:1. Preferably, the weight ratio between plasticizer and lignin and/or tannin, calculated on the basis of dry weight of each component, is from 0.1:10 to 10:10, such as from 1:10 to 5:10. The bonding resin may also comprise coupling agent. Coupling agents are for example silane-based coupling agents.

The amount of urea in the bonding resin can be 0-40% preferably 5-20% calculated as the dry weight of urea and the total weight of the bonding resin.

A filler and/or hardener can also be added to the bonding resin. Examples of such fillers and/or hardeners include limestone, cellulose, sodium carbonate, and starch.

The reactivity of the lignin can be increased by modifying the lignin by glyoxylation, etherification, esterification or any other method where lignin hydroxyl content or carboxylic content or amine content or thiol content is increased. Preferably, the lignin used according to the present invention is not modified chemically after its extraction from wood and isolation.

Preferably, the bonding resin according to the present invention does not contain formaldehyde. Preferably, the bonding resin does not contain phenol.

Preferably, the bonding resin according to the present invention does not contain basic catalyst.

The aqueous solution of lignin and/or tannin, further comprising ammonia and/or an organic base, is preferably mixed with the crosslinker at room temperature, such as at a temperature of from 15° C. to 30° C. The mixing is preferably carried out for about 5 seconds to 2 hours. Preferably, the viscosity of the mixture is monitored during mixing, either continuously or by taking samples and determining the viscosity thereof.

In the production of mineral wool insulation, curing of the bonding resin to form an adhesive takes place when the components used for the preparation of the mineral wool insulation are exposed to heating.

### EXAMPLES

### Example 1

Lignin solution was prepared first by adding 211 g of powder lignin (solid content 95%) and 685 g of water to a 1 L glass reactor at ambient temperature and stirred until the lignin was fully and evenly dispersed. Then, 104 g of 28-30% ammonia solution was added to the lignin dispersion. The composition was stirred for 60 minutes to make sure that the lignin was completely dissolved.

### Example 2

3-Aminopropyl trimethoxysilane was diluted to 1% solution in water. Binder composition was prepared by weighing 60 g of lignin-ammonia solution from the example 1, 6 g of Hydroxymethyl furfural and 9 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 1.

TABLE 1
Flexural Strength of the sand bars with and without conditioning

| | Flexural Strength | Flexural Strength after |
|---|---|---|
| | without conditioning | conditioning |
| | [MPa] | [MPa] |
| Sand bars | 4.6 | 3.2 |

### Example 3

3-Aminopropyl trimethoxysilane was diluted to 1% solution in water. Binder composition was prepared by weighing 54 g of lignin-ammonia solution from the example 1, 5.4 g of Hydroxymethyl furfural, 2.5 g water and 8.1 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm. Sand bars were post-cured for 24 hours and soaked in a water bath at 80° C. for 2 hours.

The sand bars were evaluated with 3-point bending test. The flexural strength before and after water soaking is given in the Table 2.

TABLE 2
Flexural Strength of the sand bars with and without conditioning

| | Flexural Strength | Flexural Strength after |
|---|---|---|
| | without conditioning | conditioning |
| | [MPa] | [MPa] |
| Sand bars | 4.0 | 5.0 |

### Example 4

3-Aminopropyl trimethoxysilane was diluted to 1% solution in water. Binder composition was prepared by weighing 58 g of lignin-ammonia solution from the example 1, 8.7 g of Hydroxymethyl furfural, 6 g water and 3.6 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm. Sand bars were post-cured for 24 hours and soaked in a water bath at 80° C. for 2 hours.

The sand bars were evaluated with 3-point bending test. The flexural strength before and after water soaking is given in the Table 3.

TABLE 3
Flexural Strength of the sand bars with and without conditioning

| | Flexural Strength | Flexural Strength after |
|---|---|---|
| | without conditioning | conditioning |
| | [MPa] | [MPa] |
| Sand bars | 4.8 | 3.8 |

### Example 5

3-Aminopropyl trimethoxysilane was diluted to 1% solution in water. Binder composition was prepared by weighing 57 g of lignin-ammonia solution from the example 1, 5.7 g of Hydroxymethyl furfural, 1.7 g sorbitol polyglycidyl ether, 6 g water and 3.6 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 4.

TABLE 4
Flexural Strength of the sand bars with and without conditioning

| | Flexural Strength | Flexural Strength after |
|---|---|---|
| | without conditioning | conditioning |
| | [MPa] | [MPa] |
| Sand bars | 7.8 | 6.1 |

### Example 6

3-Aminopropyl trimethoxysilane was diluted to 1% solution in water. Binder composition was prepared by weighing 57 g of lignin-ammonia solution from the example 1, 5.7 g of Hydroxymethyl furfural, 1.7 g polyglycerol polyglycidyl ether, 6 g water and 3.6 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 5.

TABLE 5
Flexural Strength of the sand bars with and without conditioning

| | Flexural Strength | Flexural Strength after |
|---|---|---|
| | without conditioning | conditioning |
| | [MPa] | [MPa] |
| Sand bars | 7.0 | 6.2 |

### Example 7

3-Aminopropyl trimethoxysilane was diluted to 1% solution in water. Binder composition was prepared by weighing 57 g of lignin-ammonia solution from the example 1, 5.7 g of Hydroxymethyl furfural, 1.7 g polyethyleneglycol diglycidyl ether, 6 g water and 3.6 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 6.

TABLE 6
Flexural Strength of the sand bars with and without conditioning

| | Flexural Strength | Flexural Strength after |
|---|---|---|
| | without conditioning | conditioning |
| | [MPa] | [MPa] |
| Sand bars | 7.0 | 6.2 |

### Example 8

3-Aminopropyl trimethoxysilane was diluted to 1% solution in water. Binder composition was prepared by weighing 57 g of lignin-ammonia solution from the example 1, 5.7 g of furfuryl alcohol, 1.7 g polyethyleneglycol diglycidyl ether, 6 g water and 3.6 g of 1% of 3-aminopropyl trimethoxysilane into a 250 ml plastic container and was stirred with a wooden stick for 2 minutes. Then, 450 g silica sand was weighed into a bowl and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 7.

TABLE 7
Flexural Strength of the sand bars with and without conditioning

| | Flexural Strength | Flexural Strength after |
|---|---|---|
| | without conditioning | conditioning |
| | [MPa] | [MPa] |
| Sand bars | 2.9 | 3.4 |

### Example 9

3-Aminopropyl trimethoxysilane was diluted to 1% solution in water. Binder composition was prepared by weighing 71 g of lignin-ammonia solution from the example 1, 2.1 g of Hydroxymethyl furfural, 2.1 g polyethyleneglycol diglycidyl ether and 3.9 g of 1% of 3-aminopropyl trimethoxysilane into a 250 ml plastic container and was stirred with a wooden stick for 2 minutes. Then, 450 g silica sand was weighed into a beaker and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 8.

TABLE 8
Flexural Strength of the sand bars with and without conditioning

| | Flexural Strength | Flexural Strength after |
|---|---|---|
| | without conditioning | conditioning |
| | [MPa] | [MPa] |
| Sand bars | 4.2 | 2.9 |

### Example 10

3-Aminopropyl trimethoxysilane was diluted to 1% solution in water. Binder composition was prepared by weighing 75 g of lignin-ammonia solution from the example 1, 0.75 g of Hydroxymethyl furfural, 2.25 g polyethyleneglycol diglycidyl ether and 3.9 g of 1% of 3-aminopropyl trimethoxysilane into a 250 ml plastic container and was stirred with a wooden stick for 2 minutes. 450 g silica sand was weighed into a bowl and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm.

Sand bars were post-cured for 24 hours and soaked in a water bath at 80° C. for 2 hours.

The sand bars were evaluated with 3-point bending test. The flexural strength before and after water soaking is given in the Table 9.

TABLE 9
Flexural Strength of the sand bars with and without conditioning

| | Flexural Strength | Flexural Strength after |
|---|---|---|
| | without conditioning | conditioning |
| | [MPa] | [MPa] |
| Sand bars from the | 2.4 | 2.2 |
| Example 10 | | |

In view of the above detailed description of the present invention, other modifications and variations will become apparent to those skilled in the art. However, it should be apparent that such other modifications and variations may be affected without departing from the spirit and scope of the invention.

1. A method for preparing a bonding resin, the method comprising:
mixing an aqueous solution comprising lignin, or tannin and ammonia, or an organic base, or any combination thereof with hydroxymethylfurfural, furfural, furfuryl alcohol, acetoxymethyl furfural, an oligomer of hydroxymethylfurfural, or a combination thereof, and optionally with one or more additives, and optionally with one or more crosslinkers selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, a diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate.

2. A method for preparing a bonding resin, the method comprising:
mixing an aqueous solution of lignin comprising ammonia, an organic base, or both with hydroxymethylfurfural, furfural, furfuryl alcohol, acetoxymethyl furfural, an oligomer of hydroxymethylfurfural, or a combination thereof, and optionally with one or more additives, and optionally with one or more crosslinkers selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, a diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate.

3. The method according to claim 2, wherein the one or more crosslinkers are selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, and a diglycidyl ether of terminal diol having a linear carbon chain of 3-6 carbon atoms.

4. The method according to claim 2, wherein the one or more crosslinkers are selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, and a propylene glycol diglycidyl ether having 1-5 propylene glycol units.

5. The method according to claim 3, wherein the one or more crosslinkers comprise is polyglycerol polyglycidyl ether.

6. The method according to claim 1, wherein the aqueous solution comprises at least 5% by weight of lignin.

7. The method according to claim 1, wherein no epoxy-based crosslinker is used.

8. The method according to claim 1, wherein a weight ratio between lignin, calculated on a basis of dry lignin, and a total amount of hydroxymethylfurfural (HMF), furfural (FU), furfuryl alcohol (FA) or acetoxymethyl furfural is from 0:0.5:10 to 10:0.1.

9. The method according to claim 1, wherein the one or more additives are mixed with the aqueous solution, and wherein the one or more additives are selected from a group consisting of: urea, tannin, surfactant, dispersing agent, plasticizer, coupling agent, and a filler.

10. The method according to claim 1, wherein the lignin is modified by glyoxylation, etherification, esterification or any other method where lignin hydroxyl content or amine content or thiol content is increased.

11. The method according to claim 1, wherein the lignin is not chemically modified after extraction and isolation before being mixed with the aqueous solution.

12. The method according to claim 1, wherein the one or more cross-linkers have an epoxy index above 4 eq/kg.

13. A bonding resin obtained by the method of claim 1.

14. A bonding resin comprising:
an aqueous solution comprising
lignin, tannin and ammonia, an organic base, or a combination thereof, and
hydroxymethylfurfural, furfural, furfuryl alcohol, acetoxymethyl furfural, an oligomer of hydroxymethylfurfural, or a combination thereof,
and optionally further comprising:
one or more additives, and
one or more crosslinkers selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl) aniline, p-(2,3-epoxypropoxy-N,N-bis(2,3-epoxypropyl) aniline, diglycidyl ether of bis-hydroxymethylfuran, a diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate.

15. The bonding resin according to claim 14, wherein the bonding resin does not comprise epoxy-based crosslinker.

16. A method for manufacturing a laminate, mineral wool insulation, or wood product, the method comprising:
preparing a bonding resin according to the method of claim 1; and,
manufacturing the laminate, mineral wool insulation, or wood product with the bonding resin.

17. The method of claim 16, wherein the bonding resin is provided to a surface, and wherein curing of the bonding resin to form an adhesive takes place when the surface is exposed to pressure and heating.

18. A laminate, mineral wool insulation, or wood product comprising:
the bonding resin according to claim 14.
